(19) United States
(12) Patent Application Publication
(10) Pub. No.: US 2026/0145005 A1
(43) Pub. Date: May 28, 2026

(54) Tumor Positioning Method, Electronic Device, and Storage Medium

(71) Applicant: OUR UNITED CORPORATION, Shaanxi (CN)

(72) Inventors: Dalin Liu, Shaanxi (CN); Hao Yan, Shaanxi (CN)

(21) Appl. No.: 19/402,239

(22) Filed: Nov. 26, 2025

(30) Foreign Application Priority Data
Nov. 28, 2024 (CN) 202411730774.X

(51) Int. Cl. A61N 5/10; G06T 5/30; G06T 5/70; G06T 7/11; G06T 7/12; G06T 7/155; G06T 7/174; G06T 7/20

(52) U.S. Cl. CPC A61N 5/1037; G06T 5/30; G06T 5/70; G06T 7/11; G06T 7/12; G06T 7/155; G06T 7/174; G06T 7/20; G06T 2207/10081; G06T 2207/20036; G06T 2207/20112; G06T 2207/20192; G06T 2207/20204; G06T 2207/30061; G06T 2207/30096

(57) ABSTRACT

The present disclosure provides a tumor positioning method, an electronic device, and a storage medium. The method includes acquiring a plan image and a real-time image of a reference object that are associated with a motion trajectory of a to-be-positioned tumor; determining boundary mask information of the reference object according to the plan image; determining deformation information of the reference object according to the plan image and the real-time image; determining a motion trajectory of the reference object according to the boundary mask information and the deformation information; and positioning and tracking the to-be-positioned tumor according to the motion trajectory of the reference object.

### CROSS REFERENCE TO RELATED APPLICATIONS

This application claims priority to Chinese Patent Application No. 202411730774.X, filed Nov. 28, 2024, the disclosure of which is hereby incorporated by reference in its entirety.

### BACKGROUND OF THE INVENTION

### Field of the Invention

The present disclosure relates to the field of medical technologies, and in particular, to the field of radiotherapy technologies, and specifically to a tumor positioning method, an electronic device and a storage medium.

### Description of Related Art

Currently, one of the key technologies in radiotherapy is to maintain precise positioning of a tumor during treatment. In particular, for tumors that move with breathing, such as lung, liver, and pancreatic tumors, the extremely low contrast of the tumor in projection images and interference from overlapping surrounding tissues, it is extremely difficult to accurately position the tumor at every moment.

In the process of tumor positioning, some other human tissues in the projection images and except the tumor (which may be referred to as substitutes, reference objects, surrogates, etc.), such as the diaphragm and chest wall, etc., usually have good contrast and clarity. Furthermore, their motion trajectories are highly correlated with the motion of tumors; therefore, these substitutes may be used for tumor positioning and tracking.

### SUMMARY OF THE INVENTION

In a first aspect, the present disclosure provides a tumor positioning method, including: acquiring a plan image and a real-time image of a reference object that are associated with a motion trajectory of a to-be-positioned tumor; determining boundary mask information of the reference object according to the plan image; determining deformation information of the reference object according to the plan image and the real-time image; determining a motion trajectory of the reference object according to the boundary mask information and the deformation information; and positioning and tracking the to-be-positioned tumor according to the motion trajectory of the reference object.

In a second aspect, the present disclosure further provides an electronic device. The electronic device includes: a processor and a memory configured to store instructions executable for the processor; where the processor is configured to execute the instructions to implement any tumor positioning method in the above first aspect.

In a third aspect, the present disclosure further provides a non-volatile storage medium, where the storage medium has stored a computer program thereon, and the computer program, when read and executed, any tumor positioning method in the above first aspect is implemented.

### BRIEF DESCRIPTION OF THE DRAWINGS

The drawings are used to better understand the present solutions and do not constitute a limitation on the present disclosure. Herein:

FIG. 1 is a scene schematic diagram of a tumor positioning system, provided by the embodiments of the present disclosure.

FIG. 2 is a schematic flowchart of a tumor positioning method, provided by the embodiments of the present disclosure.

FIG. 3 is a schematic flowchart of another tumor positioning method, provided by the embodiments of the present disclosure.

FIG. 4 is a scene schematic diagram of a tumor positioning method, provided by the embodiments of the present disclosure.

FIG. 5 is a schematic flowchart of yet another tumor positioning method, provided by the embodiments of the present disclosure.

FIG. 6 is a schematic flowchart of yet another tumor positioning method, provided by the embodiments of the present disclosure.

FIG. 7 is a schematic flowchart of yet another tumor positioning method, provided by the embodiments of the present disclosure.

FIG. 8 is a schematic flowchart of yet another tumor positioning method, provided by the embodiments of the present disclosure.

FIG. 9 is a structural schematic diagram of a tumor positioning apparatus, provided by the embodiments of the present disclosure.

FIG. 10 is a schematic block diagram of an electronic device, provided by the embodiments of the present disclosure.

### DESCRIPTION OF THE INVENTION

The technical solutions in the embodiments of the present disclosure will be described clearly and completely below with reference to the drawings in the embodiments of the present disclosure. Obviously, the described embodiments are merely some but not all of the embodiments of the present disclosure. All other embodiments obtained by those skilled in the art based on the embodiments of the present disclosure without paying any creative effort shall fall within the protection scope of the present disclosure.

It should be noted that, since the method of the embodiments of the present disclosure is executed in a tumor processing device, the respective processed objects of the tumor processing device exist in the form of data or information. For example, time is time information actually. It can be understood that if size, quantity/number, position/location, etc., are mentioned in the following embodiments, it means that data corresponding to them exist, so that the tumor processing device may process them. Specific details will not be repeated herein.

As described in the background, during the process of tumor positioning, some other human tissues in a projected image and except the tumor (which may be referred as substitutes, reference objects, surrogates, etc.), such as a diaphragm, a chest wall, etc., usually have good contrast and clarity. Furthermore, motion trajectories thereof are highly correlated with the motion of tumors; therefore, these substitutes may be used for tumor positioning and tracking.

A general substitute tracking method usually is based on gradient information of acquired images, uses polynomial fitting to fit boundaries of the substitute, so as to determine a motion trajectory of the substitute, and then performs the positioning and tracking of the tumor. However, the boundaries of the polynomial fitting substitute need to be manually initialized with polynomial coefficients. Furthermore, when performing tumor positioning and tracking, it is necessary to train a prior model of the motion trajectory of the substitute, which is cumbersome and has poor stability, resulting in relatively low efficiency and accuracy of tumor positioning.

Based on the above technical problems, the embodiments of the present disclosure provide a tumor positioning method, which may determine boundary mask information of a reference object according to a plan image, and determine deformation information of the reference object according to the plan image and a real-time image. Next, the motion trajectory of the reference object may be determined according to the boundary mask information and the deformation information, so as to position and track a to-be-positioned tumor according to the motion trajectory of the reference object subsequently. Thus, the present disclosure does not need to initialize polynomial coefficients through manual intervention, nor does it need to train a prior model, which improves the efficiency and accuracy of tumor positioning.

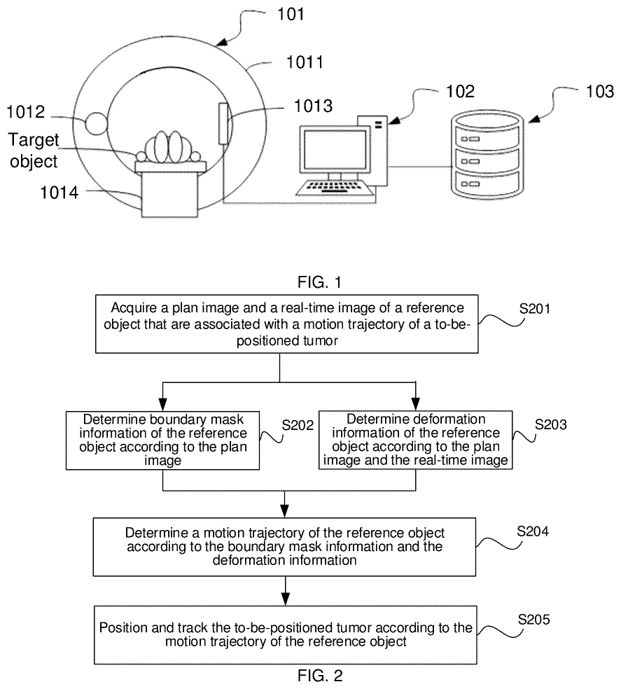

The tumor positioning method described above may be applied to a tumor positioning system. FIG. 1 is a scene schematic diagram of a tumor positioning system, provided by the embodiments of the present disclosure. The tumor positioning system may include: an image acquisition device 101, a tumor positioning device 102, and a storage server 103.

Herein, the image acquisition device 101 is a device used to acquire tumor site and surrounding normal tissues of a target object (such as a to-be-treated patient, an experimental subject, a phantom, etc.). In some embodiments, the image acquisition device 101 may be a cone-beam computed tomography (Cone—Beam Computed Tomography, CBCT) device.

Referring to FIG. 1, the image acquisition device 101 may include a gantry 1011, a tube 1012, a detector 1013, and a support apparatus 1014. Herein, the gantry 1011 may be a rotatable gantry. The tube 1012 may be used to emit imaging rays, such as kilovolt (KV) level X-ray (ray). The detector 1013 may be a detector plate (such as an X-ray detector plate) facing the tube 1012. The support apparatus 1014 is used to support and move the target object, and may be a treatment bed.

In some embodiments, when the target object is on the support apparatus 1014, the rotation of the gantry 1011 may drive the tube 1012 to project 360 degrees around the target object. After the imaging rays pass through the target object, it may be projected onto the detector 1013. At this time, the detector 1013 may acquire projection data after the projection by the tube 1012. Furthermore, after acquiring projection data from multiple projections, medical images (such as CBCT images) of the target object may be obtained through data reconstruction.

In the embodiments of the present disclosure, the image acquisition device 101 is used to acquire real-time images of a reference object in the target object. Furthermore, the real-time images may be uploaded to the tumor positioning device 102 so that the tumor positioning device 102 may perform subsequent tumor positioning method based on the real-time images.

The tumor positioning device 102 is used to determine the motion trajectory of the reference object in the target object according to the real-time images acquired by the image acquisition device 101 and plan images obtained from the storage server 103, and then position and track the to-be-positioned tumor in the target object according to the motion trajectory of the reference object.

In some embodiments, the tumor positioning device 102 may be a computer device with a graphical user interface (Graphical User Interface, GUI), and the computer device includes: one or more processors, a memory, and one or more application programs. Exemplarily, the tumor positioning device 102 may include a tumor positioning system application, and the processor of the tumor positioning device executes the tumor positioning system application to implement the tumor positioning method provided in the embodiments of the present disclosure.

In the embodiments of the present disclosure, an entity of the tumor positioning device 102 may be a terminal or a server with a display, which is not limited to the embodiments of the present disclosure.

In some embodiments, the above-mentioned terminal may be at least one of a smart phone, a smart watch, a desktop computer, a handheld computer, a virtual reality terminal, an augmented reality terminal, a wireless terminal, a laptop portable computer and other devices.

In some embodiments, the above server may be an image server. The image server is used to provide background services for the above image acquisition device 101. In some embodiments, the above-mentioned image server may be an independent physical server, or a server cluster composed of a plurality of physical servers or a distributed file system, or a cloud server that provides at least one of basic cloud computing services such as cloud services, cloud databases, cloud computing, cloud functions, cloud storage, network services, cloud communications, middleware services, domain name services, security services, content distribution networks, and big data or artificial intelligence platforms, which are not limited in the embodiments of the present disclosure. In some embodiments, a number of the above-mentioned image servers may be more or less, which is not limited in the embodiments of the present disclosure. Of course, the image server can also include other functions to provide more comprehensive and diverse services.

Furthermore, in some embodiments, the tumor positioning device 102 may run a computer system, the computer system includes a processor, and the processor is configured to implement the tumor positioning method provided by the embodiments of the present disclosure.

In some embodiments, the tumor positioning device 102 may also be connected to a radiotherapy device so that the radiotherapy device may adjust the position of the target object on the radiotherapy device according to the motion trajectory of the reference object displayed by the tumor positioning device 102.

In some embodiments, an implementation environment where the tumor positioning method is located may also include a gamma knife treatment head, an accelerator treatment head, or other radiotherapy heads, and the radiotherapy heads may be disposed on a gantry and used to emit therapeutic rays, such as gamma rays or MV-level X-rays. Thus, the implementation environment constitutes a radiotherapy system. Correspondingly, the tumor positioning device 102 is also used to perform an image registration process based on the real-time images acquired by the image acquisition device 101, and then to adjust the setup of the target object before treatment or provide real-time image guidance during treatment based on the image registration results.

The storage server 103 is used to store medical data of the target object, such as a case type of the target object, a radiotherapy plan (including the plan image of the reference object in the target object), a radiotherapy dose, etc. In the embodiments of the present disclosure, the storage server 103 may also be used to store real-time images acquired by the image acquisition device 101 and motion trajectories of reference objects generated by the above image server 102. When the tumor positioning device 102 performs the tumor positioning method provided in the embodiments of the present disclosure, the storage server 103 may send a plan image of the reference object of the target object to the tumor positioning device 102, so that the tumor positioning device 102 may execute the subsequent tumor positioning method based on the plan image.

The tumor positioning method provided by the embodiments of the present disclosure will be introduced below based on the tumor positioning system shown in FIG. 1.

The tumor positioning method provided by the embodiments of the present disclosure is performed by the tumor positioning device 102 in FIG. 1. FIG. 2 shows a flowchart schematic diagram of a tumor positioning method, provided by the embodiments of the present disclosure. As shown in FIG. 2, the tumor positioning method includes: S201 to S205.

In S201, a tumor positioning device acquires a plan image and a real-time image of a reference object that are associated with a motion trajectory of a to-be-positioned tumor.

As described in the background, if the tumor positioning is performed only based on the real-time image of the reference object, the efficiency and accuracy are relatively low. In the embodiments of the present disclosure, the tumor positioning device can acquire not only the real-time image of the reference object, but also the plan image of the reference object. In this way, by comparing the plan image and the real-time image, the motion trajectory of the reference object may be determined quickly and accurately, and thus the tumor positioning may be performed quickly and accurately.

In some embodiments, the plan image may be a CT image. Compared to the two-dimensional KV projection image, the CT image is a three-dimensional image, which is more conducive to the segmentation of reference objects (such as the diaphragm and other tissues).

In some embodiments, the reference object includes at least one of: a diaphragm, a lung wall, or a lung apex. The motion trajectory of the reference object and the motion trajectory of the to-be-positioned tumor are usually strongly correlated, that is, they have a correlation. In this way, by determining the motion trajectory of the reference object that is associated with the motion trajectory of the to-be-positioned tumor, the positioning and tracking of the tumor may be implemented.

In S202, the tumor positioning device determines boundary mask information of the reference object according to the plan image.

Specifically, when the reference object moves, its boundary usually changes, i.e., the motion trajectory of the reference object is usually strongly correlated with the boundary of the reference object. For example, when the reference object is the diaphragm, the diaphragm moves with the body's breathing. When a person inhales, the diaphragm contracts and the diaphragmatic dome (i.e., the upper boundary of the diaphragm) descends; when a person exhales, the diaphragm relaxes and the diaphragmatic dome ascends. Therefore, the tumor positioning device, after acquiring the plan image, may determine the boundary mask information of the reference object according to the plan image, so as to determine the motion trajectory of the reference object subsequently.

In S203, the tumor positioning device determines deformation information of the reference object according to the plan image and the real-time image.

Specifically, during the treatment process, the reference object of the human body may undergo deformation. When the reference object is deformed, its shape will also change accordingly. Therefore, the deformation condition of the reference object may be determined by monitoring the position change and the distance change of the reference object. Therefore, the tumor positioning device, after acquiring the plan image and the real-time image, may determine the deformation information of the reference object according to the plan image and the real-time image, so as to accurately determine the motion trajectory of the reference object subsequently.

In some embodiments, the tumor positioning device may compare the plan image and the real-time image, to determine the deformation information of the reference object.

Exemplarily, assuming the plan image is a CT image and the real-time image is a KV image, since the CT images are typically used to reconstruct the three-dimensional image, the tumor positioning device may acquire a digitally reconstructed radiograph (Digitally Reconstructed Radiograph, DRR) projection of the CT images.

Since the KV images are usually based on a sequence image from multiple angles, for each angle of KV image, the tumor positioning device, may acquire the KV image and DRR projection at that angle, determine a two-dimensional deformation field of the KV image and DRR projection at that angle, and then obtain a deformation field image corresponding to the two-dimensional deformation field. Deformation information of the reference object may be obtained through the deformation field image.

In S204, the tumor positioning device determines a motion trajectory of the reference object according to the boundary mask information and the deformation information.

Specifically, since the boundary mask information may clearly outline a contour of the reference object, positions of multiple key feature points on a boundary of the reference object may be determined through the boundary mask information of the reference object. These feature points may be vertices on the boundary of the reference object, or points with significant curvature changes, etc.

When the reference object is deformed, the shape of its boundary mask will also change accordingly. Therefore, a local deformation condition of the reference object may be inferred by monitoring the relative position change and the distance change between respective feature points on the boundary mask.

As time goes on, the above process of the position update is repeated continuously. The tumor positioning device may record a position (determined by the boundary mask and the deformation information) of the reference object at each moment, to form a sequence of positions. This sequence of positions may be used to construct the motion trajectory of the reference object.

In some embodiments, the tumor positioning device may use methods such as curve fitting, for example, polynomial fitting, spline fitting, etc., to connect these discrete position points into a continuous trajectory curve, thereby clearly showing the motion trajectory of the reference object.

In some embodiments, the tumor positioning device may also acquire other assistance information of the reference object, such as texture features inside the reference object and its relative positional relationship with surrounding tissues, etc. For example, the change direction in the internal texture of the reference object may assist the tumor positioning device in determining the motion direction of the reference object and the relative displacement between the reference object and surrounding fixed tissues, thereby further correcting the motion trajectory and improving the accuracy of the motion trajectory determination.

In S205, the tumor positioning device positions and tracks the to-be-positioned tumor according to the motion trajectory of the reference object.

Specifically, when positioning and tracking the to-be-positioned tumor according to the motion trajectory of the reference object, the tumor positioning device may first establish a relative position relationship between the reference object and the to-be-positioned tumor. For example, the tumor positioning device may determine the relative position between the reference object and the to-be-positioned tumor through medical images (such as CT, MRI, etc.). The tumor positioning device may achieve the determination of the above relative position by marking information such as the central coordinates and boundary ranges of the reference object and the to-be-localized tumor on the images.

Next, the tumor positioning device may determine a fixed geometric relationship between the reference object and the to-be-positioned tumor. For example, the reference object and the to-be-positioned tumor are connected and contain certain angles and distances.

The tumor positioning device, after determining the motion trajectory of the reference object, may convert the motion trajectory of the reference object into a potential motion trajectory of the to-be-positioned tumor according to the previously established relative position relationship.

In some embodiments, the tumor positioning device may also use operations, such as coordinate transformation and geometric translation, rotation, etc., to implement the conversion of the motion trajectory of the reference object into the potential motion trajectory of the to-be-positioned tumor. For example, if the motion trajectory of the reference object is represented by a series of coordinate points “(X, Y, Z)” in the Cartesian coordinate system, and a position offset of the to-be-positioned tumor relative to the reference object is “(Δa, Δb, Δc)”, then a motion trajectory point of the to-be-positioned tumor may be represented as (X+Δa, Y+Δb, Z+Δc).

In some embodiments, since physiological motions (such as breathing, heartbeat, etc.) of the human body have a periodic impact on the positions of the to-be-positioned tumor and the reference object, the tumor positioning device may also analyze laws of these physiological motions, separate or integrate the physiological motions with the motion trajectory of the reference object, thereby accurately determining the motion trajectory of the reference object.

For example, assuming that the motion trajectory of the reference object contains the displacement caused by breathing motion, and the motion of the to-be-positioned tumor is correlated with breathing motion (such as synchronous movement), the tumor positioning device may determine the position of the to-be-positioned tumor by analyzing breathing signals (such as acquired by the breathing belt) according to different stages of the breathing cycle, thereby improving the accuracy of positioning and tracking.

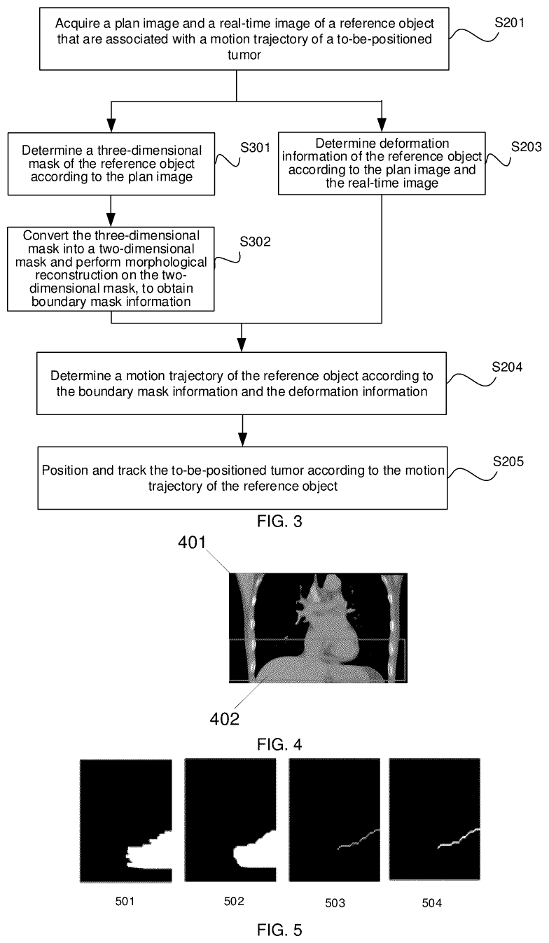

In some embodiments, referring to FIG. 2 and as shown in FIG. 3, in the above S202, the method of the tumor positioning device determining the boundary mask information of the reference object according to the plan image specifically includes the following contents.

In S301, the tumor positioning device determines a three-dimensional mask of the reference object according to the plan image.

Specifically, in order to accurately determine boundaries of the reference object and thus determine the motion trajectory of the reference object, the tumor positioning device may first determine the three-dimensional mask of the reference object according to the plan image.

In some embodiments, the plan image may include multiple layers of plan sub-images of the reference object continuously acquired along a preset anatomical direction. In this case, the method of the tumor positioning device determining the three-dimensional mask of the reference object according to the plan image specifically includes that: for each layer of plan sub-image of the multiple layers of plan sub-images, the tumor positioning device may determine a reference region to which the reference object belongs according to the plan sub-image, and perform polynomial fitting on gradient information of the reference region in the plan sub-image, to obtain the boundary information of the reference object in the plan sub-image. Subsequently, the tumor positioning device may combine the boundary information of the reference object corresponding to the each layer of plan sub-image, to obtain the three-dimensional mask of the reference object.

Exemplarily, as shown in FIG. 4, taking an example in which the plan image is a CT image and the reference object is the diaphragm, the preset anatomical direction may be a direction of the coronal plane. In this case, the plan image may include multiple coronal plane images (i.e., multiple layers of plan sub-images). For each coronal plane image, the tumor positioning device may acquire the reference region to which the diaphragm belongs in each coronal plane image, that is, the lung cavity 401.

In some embodiments, the tumor positioning device may directly load a contour of the human lung according to the coronal plane image, to obtain the lung cavity 401.

In some embodiments, the tumor positioning device may also load an outer contour mask1 of the human body according to the coronal plane image. Then, the tumor positioning device may determine the regions in the coronal plane image that are less than an air threshold (e.g., −800 HU (Henry units)) as an air mask2. Subsequently, the tumor positioning device may determine an intersection of mask1 and mask2 as the lung cavity 401.

After obtaining the reference region to which the diaphragm belongs, that is the lung cavity 401, the tumor positioning device may determine a bottom of the lung cavity as an initial range of the diaphragm in a head-feet direction, that is, an initial range of the diaphragm 402 shown in FIG. 4.

Next, within the initial range 402 of the diaphragm, the tumor positioning device may determine gradient information in the head-feet direction, that is, the adjacent pixels are subtracted in the head-feet direction within the initial range 402 of the diaphragm (a lower pixel value is subtracted from an upper pixel value in the head-feet direction). If the result of subtracting the upper pixel value from the lower pixel value is a positive number, then the gradient value is positive. This means that in the head-feet direction, the pixel value decreases, and the upper pixels are brighter than the lower pixels (assuming it is a grayscale image, the larger the pixel value, the brighter it is). Conversely, if the result of subtracting the lower pixel value from the upper pixel value is negative, then the gradient value is negative. This means that in the head-feet direction, the pixel value increases, and the upper pixels are darker than the lower pixels.

The tumor positioning device, after obtaining the gradient information, may determine the mask with a gradient value greater than a preset threshold as the boundary mask of the diaphragm, and perform polynomial fitting on the boundary mask of the diaphragm, to obtain a boundary of the diaphragm (a left side and a right side of the diaphragm), that is, the boundary information of the reference object.

Subsequently, the tumor positioning device may combine the boundary of the diaphragm corresponding to each coronal plane image, to obtain a three-dimensional mask of the diaphragm.

Of course, the tumor positioning device may also use an artificial intelligence (AI) technology, to determine the three-dimensional mask of the reference object.

In S302, the tumor positioning device converts the three-dimensional mask into a two-dimensional mask and performs morphological reconstruction on the two-dimensional mask, to obtain boundary mask information.

Specifically, after obtaining the three-dimensional mask of the reference object, since the boundary mask information of the reference object is in a two-dimensional space, the tumor positioning device may convert the three-dimensional mask into a two-dimensional mask and perform the morphological reconstruction on the two-dimensional mask, to obtain the boundary mask information.

The morphological reconstruction is an important operation in mathematical morphology. It is performed based on a mask image, and its purpose is to reconstruct a new image from a marked image under constraints of the mask image through morphological operations.

In the embodiments of the present disclosure, during the process where the tumor positioning device determines the three-dimensional mask of the reference object according to the plan image, since the plan image may have breathing artifacts (i.e., the positions of the organ may be different at different scanning time points, resulting in artifacts such as blurring, ghosting or deformation on the image), the two-dimensional mask obtained by the three-dimensional mask conversion may have false boundaries. In this case, the false boundaries in the two-dimensional mask may be removed through morphological reconstruction, to obtain accurate and clear boundary mask information.

In some embodiments, the method of the tumor positioning device performing the morphological reconstruction on the two-dimensional mask, to obtain the boundary mask information specifically includes that: the tumor positioning device may sequentially perform an erosion processing and a smoothing processing on the two-dimensional mask, to obtain a processed two-dimensional mask, and determine a boundary mask of the reference object in a first direction according to the gradient information of the processed two-dimensional mask. Subsequently, the tumor positioning device may perform a dilation processing on the reference object in a second direction based on the boundary mask and a structural element of the reference object in the first direction, to obtain the boundary mask information.

Continuing with taking an example of the reference object as the diaphragm, as shown in FIG. 5, the tumor positioning device may first perform an erosion processing on the two-dimensional mask of the diaphragm, i.e., an erosion operation, to obtain a two-dimensional mask 501 after the erosion operation is performed.

The purpose of the erosion operation is to eliminate small protrusions in the two-dimensional mask, and these small protrusions are most likely false boundaries caused by breathing artifacts. The erosion operation is performed on the two-dimensional mask by selecting appropriate structural elements (such as smaller circular structural elements or smaller square structural elements). The erosion operation causes the boundaries of the mask to shrink inward, thereby removing those discontinuous and small false-boundary portions.

Next, the tumor positioning device may perform a smoothing processing on the two-dimensional mask after the erosion operation is performed, that is, a smoothing operation (also referred to as an opening operation smoothing processing), to obtain a two-dimensional mask 502 after the smoothing processing is performed. The smoothing operation may reduce discontinuous portions at the boundaries of the two-dimensional mask, making it smoother.

Next, the tumor positioning device may determine the boundary mask 503 of the diaphragm in the first direction according to the gradient information of the processed two-dimensional mask.

Assuming that the first direction is the head-feet direction. The tumor positioning device may use pixel points with a gradient greater than 0 in the head-feet direction in the processed two-dimensional mask as boundary points, and connect and combine the boundary points, to obtain the boundary mask of the diaphragm in the head-feet direction.

Subsequently, the tumor positioning device may perform the dilation processing based on the boundary mask of the diaphragm in the head-feet direction, the dilated structuring element (i.e., the filter kernel) may be [0; 1; 1], and the dilated direction may be from the head to feet, thereby obtaining the boundary mask information 504 of the diaphragm.

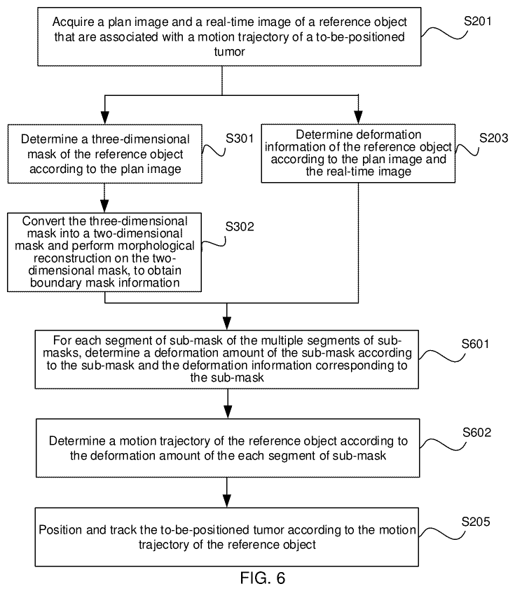

In some embodiments, the boundary mask information includes multiple segments of sub-masks of the reference object. In this case, in combination with FIG. 3 and as shown in FIG. 6, in the above S204, the method of the tumor positioning device determining the motion trajectory of the reference object according to the boundary mask information and the deformation information specifically at least includes the following contents.

In S601, for each segment of sub-mask of the multiple segments of sub-masks, the tumor positioning device determines a deformation amount of the sub-mask according to the sub-mask and the deformation information corresponding to the sub-mask.

Specifically, as can be seen from S203 above, the tumor positioning device may determine the deformation information of the reference object. For each segment of sub-mask, the tumor positioning device may acquire deformation information corresponding to the sub-mask from the deformation information. As can be seen from S302 above, the tumor positioning device may acquire the boundary mask information of the reference object, and then determine the each segment of sub-mask from the boundary mask information. Subsequently, the tumor positioning device may determine the deformation amount of the sub-mask according to the sub-mask and the deformation information corresponding to the sub-mask.

Exemplarily, assuming the boundary mask information includes n segments of sub-masks of the reference object, the deformation amount calculation formula d; for the i-th segment of sub-mask is:

$${d}_{i} = \frac{{\sum }_{x , y} m a s {k}_{i} ( x , y ) * D ( x , y )}{\sum mask ( x , y )}$$

Herein, D (x, y) is the deformation information of the pixel point (x, y), and mask (x, y) is the sub-mask of the pixel point (x, y).

In S602, the tumor positioning device determines the motion trajectory of the reference object according to the deformation amount of the each segment of sub-mask.

Specifically, after determining the deformation amount of each segment of sub-mask, the tumor positioning device may perform deformation calculation on each segment of sub-mask based on the deformation amount of the each segment of sub-mask, and generate the motion trajectory of the reference object through the sub-mask after the deformation calculation is performed.

It should be noted that, since the above operations are performed on each image in the sequences of KV images, the motion trajectory of the above reference object is also a motion trajectory generated for one KV image. The tumor positioning device may select a smoothest estimated motion trajectory from the motion trajectory corresponding to each KV image as the motion trajectory of the reference object, and determine a maximum range of its motion (e.g., a highest point, a lowest point, etc.) according to the motion trajectory of the reference object.

As can be seen from the above, by performing the segment processing on the boundary mask information, the deformation amount of each segment of sub-mask may be accurately obtained, thereby accurately determining the motion trajectory of the reference object and improving the robustness of determining the motion trajectory of the reference object.

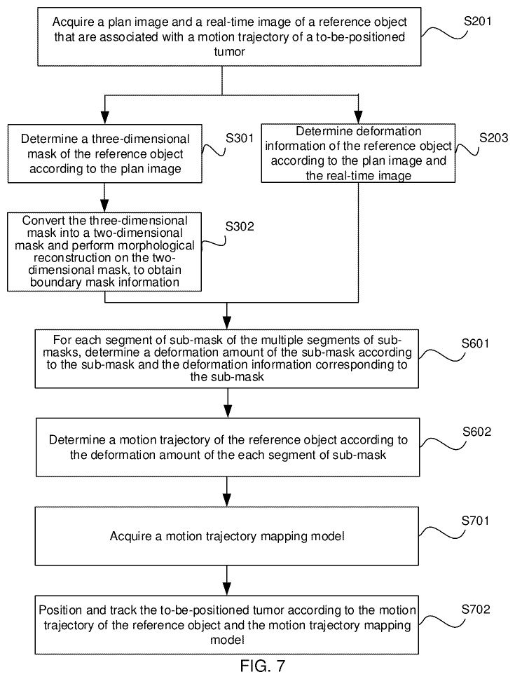

In some embodiments, in combination with FIG. 6 and as shown in FIG. 7, in the above S205, the method of the tumor positioning device positioning and tracking the to-be-positioned tumor according to the motion trajectory of the reference object specifically at least includes following contents.

In S701, the tumor positioning device acquires a motion trajectory mapping model.

Herein, the motion trajectory mapping model is used to represent a mapping relationship between the motion trajectory of the reference object and the motion trajectory of the to-be-positioned tumor.

Specifically, when generating the motion trajectory mapping model, it is necessary to acquire data related to the motion trajectory, such as the position data and motion data of the to-be-positioned tumor and the reference object.

In some embodiments, the position data of the to-be-positioned tumor may be acquired periodically through medical imaging techniques, such as CT, MRI, or PET-CT. These imaging devices may provide coordinates of the to-be-positioned tumor in the three-dimensional space, and a time interval between two scans should remain relatively stable, such as once a week or once every two weeks.

The motion data of the reference object may also be periodically acquired by using medical imaging. In the process of breathing, the position and shape of the reference object (such as the diaphragm) change. In the embodiments of the present disclosure, methods such as dynamic MRI or fluorescence fluoroscopy, etc., may be used to record the motion trajectory of the reference object during the breathing cycle and acquire its position information at different breathing phases.

After acquiring the above data related to the motion trajectory, these data may be preprocessed, to obtain processed trajectory data.

Herein, the preprocessing may include coordinate system alignment processing and time synchronization processing.

Since the position of the to-be-positioned tumor and the motion data of the reference object may come from different imaging devices or scanning sequences, it is necessary to align their coordinate systems. For example, geometric transformations, such as translation, rotation, and scaling etc., are used to ensure that all data is in a unified spatial coordinate system.

Furthermore, since the position of the to-be-positioned tumor and the motion of the reference object are processes that change over time, it is necessary to synchronize the data in time, determine each scan of the to-be-positioned tumor and time points of the motion record of the reference object, and arrange them in chronological order so as to establish an accurate mapping relationship subsequently.

Next, tumor position features and reference object motion features may be extracted from the processed trajectory data, to establish the motion trajectory mapping model.

The tumor position features may include dynamic features such as three-dimensional coordinates, the change speed of tumor position, acceleration and etc. These features may help describe the motion states of the to-be-positioned tumor at different time points, for example, the change speed of position may be obtained by calculating a difference between the tumor positions in two adjacent scans.

The motion features of the reference object may include features, such as its rise amplitude and fall amplitude, motion speed, breathing rate, etc., during the breathing cycle. For example, by taking an example in which the reference object is the diaphragm, its motion amplitude may be determined by analyzing the position difference of the diaphragm at the peak of inhalation and exhalation.

In some embodiments, when establishing the motion trajectory mapping model, if there is an approximately linear relationship between the position of the to-be-positioned tumor and the motion of the reference object, a linear regression model may be considered for use. If the relationship between the position of the to-be-positioned tumor and the motion of the reference object is complex, a nonlinear model may be required. For example, a simple multilayer perceptron (MLP) neural network may be constructed. An input layer of the neural network receives the motion features of the reference object, a hidden layer performs nonlinear transformations, and an output layer outputs the tumor position features. By training the neural network with a large amount of data and adjusting weights and biases of the network, an accurate motion trajectory mapping model may be obtained.

In S702, the tumor positioning device positions and tracks the to-be-positioned tumor according to the motion trajectory of the reference object and the motion trajectory mapping model.

In some embodiments, in a case where there are multiple reference objects, the motion trajectory mapping model may also be a mapping model between the motion trajectories of multiple reference objects and the motion trajectory of the to-be-positioned tumor. In other words, the tumor positioning device may position and track the to-be-positioned tumor according to the motion trajectories of multiple reference objects and the motion trajectory mapping model.

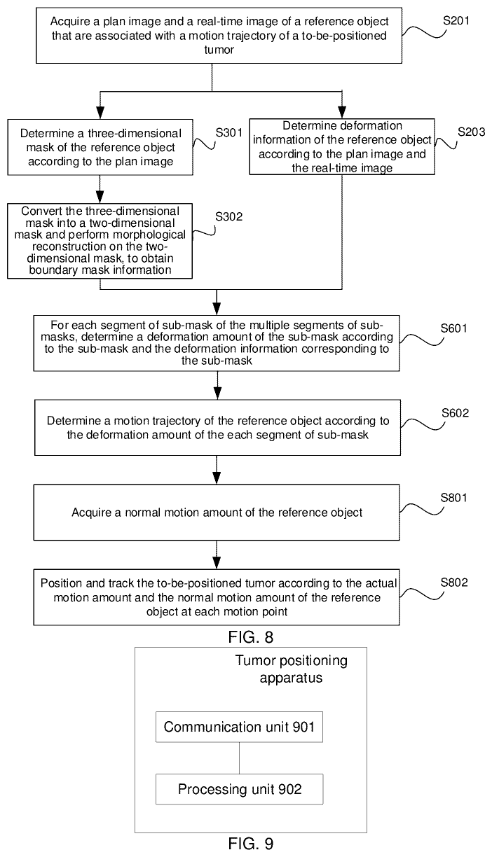

In some embodiments, the motion trajectory of the reference object includes an actual motion amount of the reference object at each motion point. In this case, in combination with FIG. 6 and as shown in FIG. 8, in the above S205, the method of the tumor positioning device positioning and tracking the to-be-positioned tumor according to the motion trajectory of the reference object specifically at least includes following contents.

In S801, the tumor positioning device acquires a normal motion amount of the reference object.

For example, by taking an example in which the reference object is the diaphragm, the motion of the diaphragm usually changes reasonably according to human breathing. In this situation, the tumor positioning device may acquire the normal motion amount of the diaphragm.

In S802, the tumor positioning device positions and tracks the to-be-positioned tumor according to the actual motion amount and the normal motion amount of the reference object at each motion point.

Continuing with the above example, if the actual motion amount (e.g., the magnitude of its ascent) of the diaphragm at a certain motion point exceeds the normal motion amount of the diaphragm, it represents that a patient's breathing is abnormal.

For another example, the tumor positioning device may set a reference baseline according to the normal motion amount of the diaphragm at each motion point. If the actual motion amount of the diaphragm of a patient exceeds a baseline range (i.e., baseline drift), it may be due to diaphragm baseline drift caused by the patient gradually relaxing as the treatment proceeds.

In both of these two cases, the tumor positioning device may output prompt information to remind doctors, to examine a target volume of the patient.

In some embodiments, in a case where there are multiple reference objects, the tumor positioning device may also acquire a normal motion range of the multiple reference objects. In the process of positioning and tracking the to-be-positioned tumor, the tumor positioning device may acquire an actual motion range of the multiple reference objects and compare the actual motion range of the multiple reference objects with the normal motion range of the multiple reference objects to judge whether actual motion ranges of respective reference objects are abnormal, thereby determining whether the breathing status of the patient is abnormal.

The tumor positioning device, when comparing the actual motion range of the multiple reference objects with the normal motion range of the multiple reference objects, may subtract the normal motion range from the actual motion range of the reference objects and judge whether the difference exceeds a preset range. Subsequently, by mapping the difference to a risk coefficient, and weighting and averaging risk coefficients of all reference objects, a final risk coefficient may be obtained and by judging whether the final risk coefficient exceeds the preset range, whether the breathing status of the patient is abnormal at this time can be judged.

The above introduces the solutions of the embodiments of the present disclosure mainly from the perspective of methods. It can be understood that, in order to implement the above functions, the tumor positioning device contains a corresponding hardware structure and/or software module for performing various functions. Those skilled in the art should easily recognize that, the embodiments of the present disclosure can be implemented in the form of hardware or a combination of hardware and computer software, in combination with the units and algorithm steps of the various examples described in the embodiments disclosed herein. Whether a certain function is executed by hardware or by computer software driving hardware, depends on the specific applications and design constraint conditions of the technical solutions. Professional technicians may use different methods to implement the described functions, for each specific application, but this implementation should not be considered beyond the scope of the embodiments of the present disclosure.

The embodiments of the present disclosure may divide the tumor positioning device into functional units according to the above method examples. For example, various functional units may be divided corresponding to the respective functions, or two or more functions may be integrated into a processing unit. The above integrated unit may be implemented in the form of hardware or in the form of a software functional unit. It should be noted that the division of the units in the embodiments of the present disclosure is schematic, which is only a logical functional division, and there may be other divisions in actual implementations.

As shown in FIG. 9, a tumor positioning apparatus is provided in the embodiments of the present disclosure and includes: a communication unit 901 and a processing unit 902;
the communication unit 901 is configured to acquire a plan image and a real-time image of a reference object that are associated with a motion trajectory of a to-be-positioned tumor;
the processing unit 902 is configured to determine boundary mask information of the reference object according to the plan image;
the processing unit 902 is further configured to determine deformation information of the reference object according to the plan image and the real-time image;
the processing unit 902 is further configured to determine a motion trajectory of the reference object according to the boundary mask information and the deformation information; and
the processing unit 902 is further configured to position and track the to-be-positioned tumor according to the motion trajectory of the reference object.

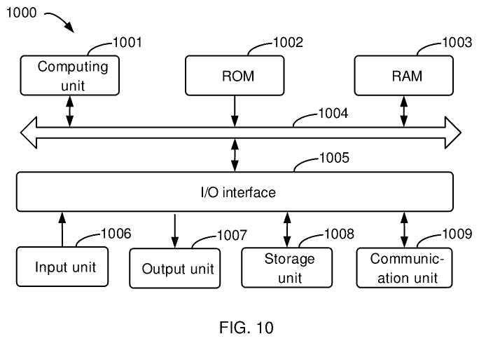

FIG. 10 shows a schematic block diagram of an example electronic device 1000 that may be used to implement the embodiments of the present disclosure. The electronic device is intended to represent various forms of digital computers, such as laptop computers, desktop computers, worktables, personal digital assistants, servers, blade servers, mainframe computers, and other appropriate computers. The electronic device may also represent various forms of mobile apparatuses, such as personal digital assistants, cell phones, smart phones, wearable devices, and other similar computing apparatuses. Herein, the shown components, connections and relationships between these components, and functions of these components are taken as examples only, and are not intended to limit the implementations of the present disclosure as described and/or claimed herein. In some embodiments, the electronic device may be the tumor positioning device shown in FIG. 1 above.

As shown in FIG. 10, the electronic device 1000 includes a computing unit 1001, which may perform various appropriate actions and processes according to a computer program stored in a read-only memory (Read-Only Memory, ROM) 1002 or a computer program loaded to a random access memory (Random Access Memory, RAM) 1003 from a storage unit 1008. Various programs and data required for the operations of the electronic device 1000 may also be stored in the random access memory 1003. The computing unit 1001, the read-only memory 1002, and the RAM 1003 are connected to each other via a bus 1004. An input/output (I/O) interface 1005 is also connected to the bus 1004.

A plurality of components in the electronic device 1000 are connected to the input/output interface 1005. The plurality of components include: an input unit 1006, such as a keyboard, a mouse, etc.; an output unit 1007, such as various types of displays, speakers, etc.; a storage unit 1008, such as a magnetic disk, an optical disk, etc.; and a communication unit 1009, such as a network card, a modem, a wireless communication transceiver, etc. The communication unit 1009 allows the electronic device 1000 to exchange information/data with other devices over a computer network and/or various telecommunication networks such as the Internet.

The computing unit 1001 may be a variety of general-purpose and/or special-purpose processing components with processing and computing capabilities. Some examples of the computing unit 1001 include, but are not limited to, a central processing unit, a graphics processing unit (GPU), various special-purpose artificial intelligence (AI) computing chips, various computing units for executing machine learning model algorithms, a digital signal processor, and any appropriate processor, controller and microcontroller, etc. The computing unit 1001 performs the various methods and processes described above, such as the imaging method. For example, in an embodiment, the imaging method may be implemented as a computer software program, which is tangibly included in a machine-readable medium, such as the storage unit 1008. In an embodiment, a part or all of the computer program may be loaded and/or installed onto the electronic device 1000 via the ROM 1002 and/or the communication unit 1009. When the computer program is loaded onto the RAM 1003 and executed by the computing unit 1001, one or more steps of the imaging method described above may be performed. Alternatively, in other embodiments, the computing unit 1001 may be configured to execute the imaging method in any other appropriate manners (e.g., by means of firmware).

The various implementations of systems and techniques described above herein may be implemented in a digital electronic circuitry system, an integrated circuitry system, a field-programmable gate array, an application-specific integrated circuit, an application-specific standard product (Application Specific Standard Part, ASSP), a system on a chip (SOC) system, a complex programmable logic device (CPLD), computer hardware, firmware, software and/or any combination thereof. The various implementations may include implementations in one or more computer programs. The one or more computer programs may be executed and/or interpreted on a programmable system including at least one programmable processor. The programmable processor may be a special-purpose or general-purpose programmable processor, may receive data and instructions from a storage system, at least one input apparatus and at least one output apparatus, and transmit the data and instructions to the storage system, the at least one input apparatus and the at least one output apparatus.

Program codes for the implementation of the method of the present disclosure may be written in any combination of one or more programming languages. These program codes may be provided for a processor or controller of a general-purpose computer, a special-purpose computer or other programmable data processing apparatuses, to cause functions/operations specified in the flowcharts and/or block diagrams to be implemented when the program codes are executed by the processor or controller. The program codes may all be executed on a machine, or partially be executed on a machine, or partially be executed on a machine and partially be executed on a remote machine as a separate software package, or all be executed on a remote machine or a server.

In the context of the present disclosure, the machine-readable medium may be a tangible medium that contains or stores a program used for an instruction execution system, apparatus or device, or a program used in conjunction with an instruction execution system, apparatus or device. The machine-readable media may be machine-readable signal media or machine-readable storage media. The machine-readable medium may include, but be not limited to, an electronic, magnetic, optical, electromagnetic, infrared or semi-conductive system, apparatus or device, or any appropriate combination thereof. More specific examples of the machine-readable storage medium may include an electrical connection based on one or more wires, a portable computer disk, a hard disk, a random-access memory, a read-only memory, an erasable programmable read-only memory, an optical fiber, a portable compact disc read-only memory, an optical storage device, a magnetic storage device, or any appropriate combination thereof.

In order to provide interactions with a user, the systems and techniques described herein may be implemented on a computer. The computer has: a display apparatus (for example, a cathode ray tube (CRT) or liquid crystal display (LCD) monitor) for displaying information to the user; and a keyboard and a pointing apparatus (for example, a mouse or a trackball), by which the user may provide an input to the computer. Other types of apparatuses may also be used to provide interactions with the user. For example, feedback provided for the user may be sensory feedback in any form (for example, visual feedback, auditory feedback or haptic feedback). Moreover, an input (including sound input, voice input or haptic input) from the user may be received in any form.

The systems and techniques described herein may be implemented in a computing system including a back-end component (for example, as a data server), a computing system including a middleware component (for example, an application server), a computing system including a front-end component (for example, a client computer with a graphical user interface or a web browser, by which the user may interact with implementations of the systems and techniques described herein), or a computing system including any combination of such a back-end component, middleware component or front-end component. The components of the system may be interconnected by any form or medium of digital data communication (for example, a communication network). Examples of the communication network include: a local area network (LAN), a wide area network (WAN) and the Internet.

The computing system may include a client and a server. The client and the server are generally remote from each other and typically interact via a communication network. A relationship between the client and the server is generated by computer programs that are running on the respective computers and have a client-server relationship with each other. The server may be a cloud server, a server of a distributed system, or a server combined with blockchains.

It should be understood that, the various forms of flows shown above may be used, with steps reordered, added or removed. For example, the various steps recorded in the present disclosure may be executed in parallel, in sequence or in a different order, as long as a desired result of the technical solutions of the present disclosure is achieved, which is not limited herein.

The above specific implementations do not constitute a limitation on the protection scope of the present disclosure. It should be understood by those skilled in the art that various modifications, combinations, sub-combinations and substitutions may be made according to design requirements and other factors. Any modifications, equivalent substitutions, improvements and the like made within the spirit and principle of the present disclosure shall fall within the protection scope of the present disclosure.

What is claimed is:

1. A tumor positioning method, comprising:
acquiring a plan image and a real-time image of a reference object that are associated with a motion trajectory of a to-be-positioned tumor;
determining boundary mask information of the reference object according to the plan image;
determining deformation information of the reference object according to the plan image and the real-time image;
determining a motion trajectory of the reference object according to the boundary mask information and the deformation information; and
positioning and tracking the to-be-positioned tumor according to the motion trajectory of the reference object.

2. The method according to claim 1, wherein determining the boundary mask information of the reference object according to the plan image comprises:
determining a three-dimensional mask of the reference object according to the plan image; and
converting the three-dimensional mask into a two-dimensional mask, and performing morphological reconstruction on the two-dimensional mask, to obtain the boundary mask information.

3. The method according to claim 2, wherein the plan image comprises multiple layers of plan sub-images of the reference object continuously acquired along a preset anatomical direction; and determining the three-dimensional mask of the reference object according to the plan image comprises:
for each layer of plan sub-image of the multiple layers of plan sub-images, determining a reference region to which the reference object belongs according to the plan sub-image;
performing polynomial fitting on gradient information of the reference region in the plan sub-image, to obtain boundary information of the reference object in the plan sub-image; and
combining the boundary information of the reference object corresponding to the each layer of plan sub-image, to obtain the three-dimensional mask of the reference object.

4. The method according to claim 2, wherein performing the morphological reconstruction on the two-dimensional mask, to obtain the boundary mask information comprises:
performing an erosion processing and a smoothing processing on the two-dimensional mask sequentially, to obtain a processed two-dimensional mask;
determining a boundary mask of the reference object in a first direction according to the gradient information of the processed two-dimensional mask; and
performing a dilation processing on the reference object in a second direction, to obtain the boundary mask information based on the boundary mask of the reference object in the first direction and a structural element.

5. The method according to claim 1, wherein the boundary mask information comprises multiple segments of sub-masks of the reference object; and determining the motion trajectory of the reference object according to the boundary mask information and the deformation information comprises:
for each segment of sub-mask of the multiple segments of sub-masks, determining a deformation amount of the sub-mask according to the sub-mask and deformation information corresponding to the sub-mask; and
determining the motion trajectory of the reference object according to the deformation amount of the each segment of sub-mask.

6. The method according to claim 1, wherein positioning and tracking the to-be-positioned tumor according to the motion trajectory of the reference object comprises:
acquiring a motion trajectory mapping model; wherein the motion trajectory mapping model is used to represent a mapping relationship between the motion trajectory of the reference object and the motion trajectory of the to-be-positioned tumor; and
positioning and tracking the to-be-positioned tumor according to the motion trajectory of the reference object and the motion trajectory mapping model.

7. The method according to claim 1, wherein the motion trajectory of the reference object comprises an actual motion amount of the reference object at each motion point; and positioning and tracking the to-be-positioned tumor according to the motion trajectory of the reference object comprises:
acquiring a normal motion amount of the reference object; and
positioning and tracking the to-be-positioned tumor according to the actual motion amount and the normal motion amount of the reference object at each motion point.

8. The method according to claim 1, wherein the reference object comprises at least one of: a diaphragm, a lung wall, or a lung apex.

9. The method according to claim 2, wherein the reference object comprises at least one of: a diaphragm, a lung wall, or a lung apex.

10. The method according to claim 3, wherein the reference object comprises at least one of: a diaphragm, a lung wall, or a lung apex.

11. The method according to claim 4, wherein the reference object comprises at least one of: a diaphragm, a lung wall, or a lung apex.

12. An electronic device, wherein the electronic device comprises:
a processor; and
a memory configured to store instructions executable for the processor;
wherein the processor is configured to execute the instructions, to implement operations of:
acquiring a plan image and a real-time image of a reference object that are associated with a motion trajectory of a to-be-positioned tumor;
determining boundary mask information of the reference object according to the plan image;
determining deformation information of the reference object according to the plan image and the real-time image;
determining a motion trajectory of the reference object according to the boundary mask information and the deformation information; and
positioning and tracking the to-be-positioned tumor according to the motion trajectory of the reference object.

13. The device according to claim 12, wherein the processor is further configured to:
determine a three-dimensional mask of the reference object according to the plan image; and
convert the three-dimensional mask into a two-dimensional mask, and performing morphological reconstruction on the two-dimensional mask, to obtain the boundary mask information.

14. The device according to claim 13, wherein the plan image comprises multiple layers of plan sub-images of the reference object continuously acquired along a preset anatomical direction; and the processor is further configured to:
for each layer of plan sub-image of the multiple layers of plan sub-images, determine a reference region to which the reference object belongs according to the plan sub-image;
perform polynomial fitting on gradient information of the reference region in the plan sub-image, to obtain boundary information of the reference object in the plan sub-image; and
combine the boundary information of the reference object corresponding to the each layer of plan sub-image, to obtain the three-dimensional mask of the reference object.

15. The device according to claim 13, wherein the processor is further configured to:
perform an erosion processing and a smoothing processing on the two-dimensional mask sequentially, to obtain a processed two-dimensional mask;
determine a boundary mask of the reference object in a first direction according to the gradient information of the processed two-dimensional mask; and
perform a dilation processing on the reference object in a second direction, to obtain the boundary mask information based on the boundary mask of the reference object in the first direction and a structural element.

16. The device according to claim 12, wherein the boundary mask information comprises multiple segments of sub-masks of the reference object; and the processor is further configured to:
for each segment of sub-mask of the multiple segments of sub-masks, determine a deformation amount of the sub-mask according to the sub-mask and deformation information corresponding to the sub-mask; and
determine the motion trajectory of the reference object according to the deformation amount of the each segment of sub-mask.

17. The device according to claim 12, wherein the processor is further configured to:
acquire a motion trajectory mapping model; wherein the motion trajectory mapping model is used to represent a mapping relationship between the motion trajectory of the reference object and the motion trajectory of the to-be-positioned tumor; and
position and track the to-be-positioned tumor according to the motion trajectory of the reference object and the motion trajectory mapping model.

18. The device according to claim 12, wherein the motion trajectory of the reference object comprises an actual motion amount of the reference object at each motion point; and the processor is further configured to:
acquire a normal motion amount of the reference object; and
position and track the to-be-positioned tumor according to the actual motion amount and the normal motion amount of the reference object at each motion point.

19. The device according to claim 12, wherein the reference object comprises at least one of: a diaphragm, a lung wall, or a lung apex.

20. A non-transitory storage medium, wherein the storage medium has stored a computer program thereon, and the computer program, when read and executed, the method according to claim 1 is implemented.
